(19) United States
(12) Patent Application Publication
(10) Pub. No.: US 2020/0060727 A1
(43) Pub. Date: Feb. 27, 2020

(54) ARTIFICIAL INSEMINATION DEVICE AND A METHOD OF PERFORMING ARTIFICIAL INSEMINATION USING THE DEVICE

(71) Applicant: MBH-INTERNATIONAL A/S, Allerød (DK)

(72) Inventor: Ana Latorre DUQUE, Alcorisa (ES)

(21) Appl. No.: 16/612,367

(22) PCT Filed: May 9, 2018
(86) PCT No.: PCT/DK2018/050100

§ 371 Date: Nov. 8, 2019

(30) Foreign Application Priority Data
May 11, 2017 (DK) PA 2017 70336

(51) Int. Cl. A61B 17/43; A61M 5/24

(52) U.S. Cl. CPC A61B 17/43; A61D 19/027; A61M 5/2422

(57) ABSTRACT

Artificial insemination device (1A, IB, IB′, IB″, IB′″, 1C, ID, IE, IF) for depositing sperm (15) at the cervix entrance of a subject, the device comprises: a longitudinal tube (3) for accommodating sperm (15), and a flexible guiding means (2A, 2B, 2B′, 2B″, 2B′″, 2C, 2D, 2E, 2F) connected to the tube (3), where the guiding means is arranged for being attached to an insertion unit (7) in order to facilitate at least partly insertion of the tube (3) into the vagina by means of the insertion unit (7).

The present invention relates to a device for artificial insemination where the insemination can be performed without a doctor or other healthcare professionals. If the subject to be inseminated is a woman, it is possible for the woman herself or e.g. her partner to perform the insemination, in which case the insemination can be performed in a reliable and comfortable place and at a convenient time chosen by the woman and/or her partner.

The insemination method for which the present invention is intended consist of depositing sperm into the vagina of the subject at the cervix entrance, where the method can be performed without requiring a clinic and/or healthcare professionals, but rather can be perform by persons without healthcare expertise.

As used herein, the term “home insemination” is defined as said insemination method. As used herein, the term “at the cervix entrance” is defined as at or close to the cervix entrance, where close to the cervix entrance e.g. could be a position inside the vagina within about 2 cm from the cervix entrance.

This kind of insemination method is easy, painless and the cervix acts as a filter for preventing any contaminated fluid from entering the uterus. The success rate of said insemination method increases when the distance between the point of ejection of the sperm and the cervix entrance decreases, and female orgasm or female sexual pleasure may increases the sperm uptake into the uterus.

Several ways to perform home insemination are known in the art. A well-known method for home insemination of a woman is the turkey baster method where a turkey baster or some other needleless syringe is used for drawing up already collected sperm, injecting the sperm into the vagina, and then hope that the sperm finds its way to the cervix of the woman. However, the turkey baster method lacks the intimate and relational properties of sexual intercourse, and may thus cause the insemination process to be stressful and/or unpleasant to some women.

A more recent method is described in US patent publication no. 2014/0200400 where a dildo with an internal channel along its longitudinal length is provided. An internal tube is placed in the channel and attached to a hub at the proximal end of the dildo, and the ends of an external tube are attached to the hub and a pumping mechanism, respectively. The modified dildo method brings the sexual act into the insemination process, but is limited to the fact that not all women would enjoy a dildo and/or a dildo with a determinate size. Another drawback of the dildo method, and other similar methods, is that even though the internal and external tubes can be thrown away after use, and replaced with new tubes that are clean, it is required to meticulously clean the inside and outside of the dildo in order to prevent infection next time the dildo is used.

It is noted that several insemination attempts are most often required before conception is achieved, which means that known devices for home insemination should be completely sterilized between every use of the device, in order to minimize the risk of infections.

With the known methods, the person operating the turkey baster, dildo or any similar element is not able to directly sense the location of the cervix entrance. Rather, said person will have to imagine where the cervix entrance is, which naturally significantly reduces the likelihood of actually depositing the sperm at the cervix entrance.

Hence, there is a need for a device for home insemination that makes is possible to perform the insemination in a comfortable way, possibly during sexual intercourse, and at the same time reduces/removes the known infection risk.

It is a first aspect of the present invention to provide an insemination device that can be used together with a variety of different insertion units.

It is a second aspect of the present invention to provide an insemination device that can be used with an insertion unit of a kind that makes the execution of the insemination comfortable for the subject.

It is a third aspect of the present invention to provide an insemination device where the user of the device can use his/her sense of touch to guide the device inside the vagina of the subject so that the point of ejection of the sperm is at or near the cervix entrance.

It is a fourth aspect of the present invention to provide an insemination device that is inexpensive to manufacture and is simple and reliable to use.

The novel and unique way whereby these and further aspects are achieved according to the present invention, is by providing an artificial insemination device comprising a longitudinal tube for accommodating sperm to be used for the insemination, and a flexible guiding means connected to the tube, wherein the guiding means is arranged for being releasable attached to an insertion unit in order to facilitate at least partly insertion of the tube into the vagina by means of the insertion unit.

As used herein, the term “insertion unit” is defined as a finger, a penis, a sexual toy such as a dildo, or some other substantially elongated element capable of being at least partly inserted into the vagina of a subject, where the length of the insertion unit is sufficient for the user/operator of the insertion unit to position the distal end of the tube at the cervix entrance.

Lesbian couples, single women without a male partner, or heterosexual couples where the male is infertile or has reduced infertility may use the present invention.

With the present invention, it is possible to use fresh sperm as well as sperm without the seminal fluid. It is also possible to use frozen sperm, in which case the sperm has to be unfreezed before use. As used herein, the term “sperm” is defined as all types of sperm.

As used herein, the term “distal end” of any element, unit or similar is defined as the end intended to be closest to the cervix of a subject during insemination. Correspondingly, the term “proximal end” of said element, unit or similar is defined as the opposite end intended to be furthest away from the cervix of said subject during insemination.

When the subject is a woman, the insemination device of the present invention allows the insemination procedure to be combined with the intimate and relational properties of sexual intercourse, because it is possible to choose an insertion unit (such as a finger or the penis of her partner; or her own finger or a sexual toy) of choice that makes the insemination procedure comfortable and/or sexual pleasant for the woman. With the known home insemination devices, the object being inserted into the vagina of a woman is a part of the manufactured insemination device, such as the specially designed dildo disclosed in US2014/0200400, that may not be comfortable and/or sexual pleasant for any women.

When the subject is an animal, such as a farm animal, it is possible to choose an insertion unit that is appropriate and comfortable for the specific animal, which e.g. depends on the shape and dimensions of the insertion unit. It would e.g. be possible for a farmer to put a plastic glove on one of his hands, and then attach the insemination device of the present invention to one of his gloved fingers, so that he is able to use his sense of touch to guide the distal end of the tube towards the cervix entrance of the animal, or possible even inside the cervix.

Naturally, the required shape and dimensions (especially the length) of an insertion unit that is appropriate for a specific subject depends on the anatomy of the specific subject, such as the distance from the vaginal opening to the cervix entrance. The insemination device of the present invention can be produced and marketed independently of the insertion unit, as the user of the device may already have an insertion unit that she/he would like the guiding means to be attached to. Thus, the insertion unit does not have a standardized shape/dimensions, but as the user has an immediate understanding of the different shapes/dimensions of different appropriate insertion units (such as a finger or a penis), it is easy for the user to make sure that the shape and dimensions of the guiding means of a specific insemination device fits the shape and dimensions of a specific insertion unit.

The tube can be an integral part of the guiding means, or embedded into the guiding means, such as if the tube and the guiding means are manufactured as one unit. Alternatively, the tube can be attached to the guiding means, such as by means of an adhesive, either during the manufacture of the insemination device or by the user prior to attaching the insemination device to an insertion unit.

Male sperm cells will only survive for a short time period at room temperature. It is thus preferred that sperm is drawn up into the tube just before the actual insemination is performed to increase the survival rate of the sperm cells. This is possible by e.g. positioning the distal end of the tube in already collected sperm in order to draw up sperm into the tube as a last step before the insemination, such as by operating a pumping mechanism attached to the proximal end of the tube. The already collected sperm can be fresh sperm collected in e.g. a cup or a vial shortly before the actual insemination. The already collected sperm can also be sperm in a sperm straw delivered from a sperm bank, in which case the distal end of the tube is attached to the sperm straw in order to draw up sperm into the tube.

When sperm has been drawn up into the tube, the insemination device is ready to perform the actual insemination of the subject, which is carried out by at least partly inserting the insertion unit into the vagina in such a way that the tube is also at least partly inserted into the vagina as the tube is connected to the guiding means that is attached to the insertion unit. While the insertion unit is inside the vagina, sperm is ejected from the tube into the vagina, such as by operating a pumping mechanism attached to the proximal end of the tube. The pumping mechanism can be permanently attached to the proximal end of the tube in a fluid tight manner, or the pumping mechanism can be adapted to be connected with the proximal end of the tube by the user.

The tube can be adapted so that its distal end can be connected directly to a sperm straw, such as when the outer diameter of the distal end of the tube is slightly smaller than the inner diameter of the sperm straw. A sperm straw is typically made of a biocompatible material such as polyethylene terephthalate glycol or ionomeric resin, and its internal diameter can e.g. be 2.5 mm or 2.25 mm. The distal end of the tube may have an outer diameter of about 2.4 mm, so said tube fits tightly inside a standard sperm straw with an inner diameter of about 2.5 mm, or its outer diameter may be 2.15 mm so that it fits tightly inside a standard sperm straw with an inner diameter of about 2.25 mm.

The insemination device may comprise a connector with an inlet and an outlet in fluid communication with the inlet, where the outlet is adapted to be connected with the distal end of the tube in a fluid tight manner and the inlet can be adapted to be connected with a sperm straw in a fluid tight manner.

The internal diameter of the inlet of the connector can be adapted so that sperm straws having a specific outer diameter fits fluid tightly inside the inlet. It is however preferred that the opening of the inlet of the connector has the shape of a truncated cone that tapers in the direction toward the outlet of the connector, so that different sperm straws with different outer diameters fits fluid tightly inside the inlet of such a connector (a sperm straw with a smaller diameter will have to be inserted further into the opening before a fluid tight connection is obtained than a sperm straw with a larger outer diameter).

In order to prevent that the user by mistake connects the outlet of the connector with the distal end of the tube in a non-fluid tight manner, the outlet of the connector may be permanently attached to the distal end of the tube in a fluid tight manner to prevent leaks when sperm is sucked into the tube, in which case the connector should be removed from the tube after sperm has been drawn up into the tube and before the insertion unit with the insemination device is inserted into the vagina, such as by cutting through the tube next to the outlet of the connector by means of e.g. scissors.

In order to prevent that the user by mistake connects the proximal end of the tube with a pumping mechanism in a non-fluid tight manner, the proximal end of the tube may be permanently connected to a pumping mechanism in a fluid tight manner to prevent leaks when sucked into the tube or pumped out of the tube.

As used herein, the term “pumping mechanism” is defined as a rubber bulb, a syringe (e.g. with a volume of 1-5 ml), a dropper, or some other suitable mechanism capable of being attached to a tube in order to draw up sperm into the tube (suction), and subsequently eject sperm from the tube (pumping). A syringe is preferred as the user may draw up sperm from several sperm straws one after the other before starting the actual insemination.

During the ejection of the sperm, the insertion unit is preferably positioned so that the distal end of the tube is as close to the cervix entrance as possible, so that the travelling distance for the ejected sperm is as short as possible in order to increase the likelihood of the sperm entering the cervix and thereby increase the likelihood of conception.

The flexible guiding means is adapted to facilitate the releasable attachment of the insemination device to an insertion unit in a way where the tube, in the attached position, extends along at least a part of the longitudinal length of the insertion unit and where the distance between the tube and the outer surface of the insertion unit preferably is as small as possible. With the knowledge of the position of the distal end of the tube in relation to the distal end of the insertion unit, the user is able to guide the distal end of the tube as close as possible to the cervix entrance by simply positioning the distal end of the insertion unit at an appropriate location inside the vagina of the subject. If the distal end of the tube is at the distal end of the insertion unit, the distal end of the tube will be at the cervix entrance when the distal end of the insertion unit is at the cervix entrance.

In a preferred embodiment, the insemination device is adapted to be attached to an insertion unit in the form of a finger of the user, and since the guiding means is flexible, the movement of the finger of the user inside the vagina will not be limited much by the insemination device, which provides a very simply and easy way of allowing a user being familiar with the location of the cervix entrance to use his/her sense of touch to locate the position of the cervix entrance in order to guide/position the distal end of the tube as close as possible to the cervix entrance during ejection of the sperm.

A lubricant may be provided on at least a part of the outer surface of the guiding means in order to facilitate the insertion of the insertion unit with the insemination device into the vagina of the subject. It is preferred that at least the part of the outer surface provided with such a lubricant is covered by protective film, where the film is removed by the user before the insemination is carried out. The purpose of such a protective film is to preserve the efficiency of the lubricant during storage of the insemination device.

The insemination device of the present invention is intended to be disposed off after a single use in order to minimize infection risks. This also means that no cleaning and/or sterilization of the insemination devices is required after use.

The tube is preferably an integral part of or embedded into the flexible guiding means, so that the tube does not detach from the guiding means during use of the insemination device. Further advantages are that the user does not have to spend time for attaching the tube to the guiding means, and it is avoided that the user for some reason happens to attach the tube to the guiding means at an inappropriate position.

The length of the tube and the shape/dimensions of the guiding means can be adapted so that the tube extends from a distal end of the guiding means to a proximal end of the guiding means, which makes is possible for the user of the insemination device to position the distal end of the tube at the cervix entrance, by simply attaching the guiding means on the insertion unit with the distal end of the guiding means at or near the distal end of the insertion unit and the proximal end of the guiding means toward the proximal end of the insertion unit. Afterwards, the user simply has to position the distal end of the insertion unit at the cervix entrance in order to automatically position the distal end of the tube at the cervix entrance. Positioning the distal end of the insertion unit at the cervix entrance, can be performed intuitively by the user by utilizing his/her knowledge of the shape of the insertion unit, and if the insertion unit is attached to one of his/her fingers, he/she can also utilize his/her sense of touch.

The length of the tube and the shape/dimensions of the guiding means can be adapted so that the tube extends beyond the distal end of the guiding means, such as by 10-150 mm. One of the advantages thereof is that already collected sperm can be drawn up from e.g. an open container even when the shape and dimensions of the opening of the container are of a kind that does not allow the distal end of the insertion unit to enter the container via said opening, as long as the diameter of the distal part of the tube fits the opening of the container. Another advantage is that it is much easier to connect the distal end of the tube to e.g. a sperm straw with already collected sperm, as the part of the tube extending beyond the distal end of the guiding means can conveniently be grabbed by the user in order to facilitate the act of attaching the tube to the sperm straw.

For embodiments where the tube extends beyond the distal end of the guiding means, the part of the tube extending beyond the distal end of the guiding means can conveniently be cut off after sperm has been drawn up into the tube, so that the distal end of the remaining tube is at the distal end of the guiding means.

The proximal end of the tube may extend beyond the proximal end of the guiding means, and the part of the tube extending beyond the proximal end of the guiding means preferably has a length adapted for allowing the proximal end of the tube to remain outside the vagina during the actual insemination. The advantage thereof is that a pumping mechanism connected to the proximal end of the tube is able to remain outside the vagina at all time for easy operation.

The flexible guiding means is preferably arranged for being able to releasable attach the insemination device to an insertion unit without damaging or in other ways permanently altering the insertion unit, as the releasable attachment e.g. is obtained due to friction between the guiding means and the insertion unit, and/or due to an adhesive material provided on the guiding means.

In one embodiment of the present invention, the flexible guiding means can be a hollow tubular sleeve having a proximal end with an opening for introducing the insertion unit into the sleeve. The distal end of said sleeve is preferably closed.

Said hollow tubular sleeve is preferably shaped and dimensions to fit tightly on the chosen insertion unit. For instance, when the insertion unit is a finger, the shape and dimensions of the sleeve preferably allows the sleeve to be placed easily on the finger, and at the same time ensures that the sleeve will not fall off during insemination.

Once placed in the intended position on the insertion unit, the sleeve may be held in place on the insertion unit due to friction between the inner surface of the sleeve and the outer surface of the insertion unit, such as if the sleeve is made from silicone and the insertion unit is a finger of the user.

The sleeve may have a securing means for further securing the sleeve to the inserting unit. Said securing means is preferably a string attached to the proximal end of the sleeve and arranged for pressing the proximal end of the sleeve towards the insertion unit by looping the string about the proximal end of the sleeve, in which position the string is tied by forming its ends into a knot.

The shape and dimensions of the hollow tubular sleeve preferably match the shape and dimensions of the chosen insertion unit in order to increase the friction between the inner surface of the sleeve and the outer surface of the insertion unit.

The hollow tubular sleeve is preferably formed with the tube being an integral part of or embedded into the sleeve, where the tube preferably extends along the entire length of the sleeve (i.e. from the proximal end of the sleeve to the distal end of the sleeve), or the tube may extend beyond the distal end and/or the proximal end of the sleeve.

For the embodiment of the hollow tubular sleeve where its distal end is closed, the sleeve may be provided with an air hole at its distal end, so that air is able to escape from the interior of the sleeve through said air hole when an insertion unit is inserted into the sleeve through the opening at the proximal end of the sleeve. By having such an air hole, it is possible for a hollow tubular sleeve to fit tightly on the insertion unit, such as a finger of the user, as the air inside the sleeve is able to escape from the interior of the sleeve through the air hole when the sleeve is fitted onto the insertion unit. Such a tight fit between the sleeve and the insertion unit increases the friction between the sleeve and the insertion unit, which keeps the sleeve in place on the insertion unit during use of the insemination device.

The flexible hollow tubular sleeve is preferably made of an elastic deformable material, such as silicone, polyurethane latex or rubber, so that the sleeve easily and conveniently can be placed on the insertion unit, and at the same time is gentle to the vaginal wall and skin of the subject to be inseminated.

In an alternative embodiment of the present invention, the flexible guiding means is a flexible sheet, where the material, shape and dimensions of the sheet are of a kind that allows the sheet to be wrapped at least partly about the insertion unit, and where the sheet is provided with releasable attachment means for releasable attachment of the sheet to the insertion unit.

The flexible sheet can have different shapes (such as rectangular, circular or triangular) and different dimensions depending on the shape/dimensions of the insertion unit to which the sheet is intended to be attached.

One of the advantages of using a guiding means in the form of such a flexible sheet is that a specific sheet having a specific shape and specific dimensions can be wrapped about and attached to different insertion units having different shapes and/or dimensions (such as different fingers with different circumferences and lengths), as long as it is possible to attach the sheet to the insertion unit with a sufficient attachment force. One on hand, it might not be necessary for the sheet to be wrapped about the entire circumference of the insertion unit. On the other hand, the sheet may overlap itself when wrapped about the insertion unit. Said attachment force is sufficient when the guiding means, during use of the insemination device, is substantially maintained in the intended position on the insertion unit.

It is preferred that the thickness of the flexible sheet is so small that the user is able to sense touch through the sheet when the insertion unit is e.g. a finger or a penis. The thickness could e.g. be 0.1 to 0.5 mm.

The flexible sheet is preferably adapted to be wrapped about an insertion unit in the form of a finger in a way where the fingertip is not covered by the sheet, so that the sense of touch of the user is increased due to the direct contact between the skin of the fingertip and the vaginal wall and the cervix entrance.

The flexible sheet may advantageously be shaped and dimensioned and otherwise adapted to be wrapped about an insertion unit in the form of a finger so that parts of the finger at or around the proximal interphalangeal joint and/or distal interphalangeal joint are left uncovered by the sheet so that one or both joints can move more easily, which makes it more comfortable for the user to use the insemination device.

The releasable attachment means of the flexible sheet may be an adhesive material provided on at least a part of the inner side of the sheet, where said inner side is the side of the sheet that faces towards the insertion unit when the sheet is wrapped about the insertion unit.

As used herein, the term “inner side” of the flexible guiding means is defined as the side of the guiding means that faces towards the insertion unit when the guiding means is in the intended attached position on the insertion unit, and the term “outer side” of the guiding means is defined as the opposite side of the sheet facing away from the insertion unit. Correspondingly, the term “inner surface” of the guiding means is defined as the surface facing towards the insertion unit, and the term “outer surface” is the surface facing away.

Adhesive material may be provided on the entire inner surface of the sheet in order to increase the attachment force. Another way of increasing said attachment force is by providing one or more bands of adhesive material on the inner surface of the sheet, where each band is a continuous or discontinuous band, and where each band preferably extends from the proximal end of the sheet to the distal end of the sheet.

The flexible guiding means may be a flexible sheet where the tube is located at one of the transverse sides of the sheet and one or more bands of adhesive material is provided at the opposite transverse side of the sheet. Such a sheet can be wrapped around an insertion unit having a smaller circumference than the transverse width of the sheet by starting the wrapping with the transverse side with the tube and ending with the transverse side with the one or more adhesive bands, so that at least a part of the sheet with the adhesive material can adhere onto the sheet itself and thereby maintaining the sheet in a wrapped position on the insertion unit during use. It is preferred that the tube and/or each of the one or more bands of adhesive material extends along the entire longitudinal length of the sheet, i.e. between the distal end and the proximal end of the sheet.

In addition to the adhesive material provided on at least a part of the inner side the sheet, the releasable attachment means of the flexible sheet may comprise one or more strips of friction generating material provided on the inner surface of the sheet and preferably extending substantially transverse in relation to the direction from the proximal end of the sheet to the distal end of the sheet, where the friction generating material is of a kind that provides a friction force between the sheet and the insertion unit so that displacement of the sheet in relation to the insertion unit, especially in the direction from the proximal end to the distal end of the insertion unit, is prevented. The strips could e.g. be made of silicone, which has an appropriate friction force against the human skin.

For the embodiments of the present invention where the guiding means is a flexible sheet, the sheet is preferably provided with at least one perforated line extending from the proximal end of the sheet to the distal end of the sheet. The purpose of the perforated line(s) is to enable the user to reduce the transverse length of the sheet by simply tearing off a part of the sheet at such a perforated line. Said elastic sheet is preferably provided with adhesive material on the entire inner surface of the sheet.

If such a sheet with at least one perforated line is to be wrapped about an insertion unit with a circumference that is smaller than the transverse width of the sheet, the transverse width of the sheet can be reduced by tearing off an appropriate part of the sheet. In this way, it is possible to reduce the degree by which the sheet overlaps itself when wrapped around an insertion unit, or even to reduce the width of the sheet so much that the sheet width is smaller than the circumference of the insertion unit so that the sheet does not overlap itself. A higher degree of overlap increases the strength by which the sheet is attached to an insertion unit, but as the sensitivity of an insertion unit in the form of e.g. a finger is reduced in the areas covered by the sheet, and even more in the areas where the sheet overlaps itself, the user can choose to reduce the transverse width of the sheet by tearing off an appropriate part of the sheet along a perforated line.

Naturally, it is also possible to reduce the transverse length of a sheet according to the present invention, with or without perforated lines, by simply cutting off an appropriate part of the sheet by means of e.g. scissors.

The tube may be attached on the inner side of the sheet, so that the tube is located between the insertion unit and the sheet when the sheet is wrapped around an insertion unit. The tube may be attached on the inner side of the sheet by means of e.g. an appropriate adhesive, or by means of at least two attachment strips with one strip at the distal end of the sheet and another strip at the proximal end of the sheet.

For the embodiments of the present invention where the guiding means is a flexible sheet, the sheet may be a laminate comprising two or more layers (such as layers of film), where each layer is made of an appropriate material, such as a hypoallergenic material that is non-reactive with the user's tissue. An appropriate material of the film layers can e.g. be plastic, silicone, polyurethane and/or latex. The layers are preferably joined on top of each other in order to provide a laminate, and the tube is advantageously located between the two or more layers and can be attached to each adjacent layer e.g. by means of an adhesive. It is preferred to have a smooth transition from the parts of the sheet without the tube to the part of the sheet where the tube is located, such as by varying the thickness of one or more of the layers in the proximity of the tube. Such a smooth transition makes the contact between the device of the present invention and the vagina more pleasant.

For the embodiments of the present invention where the guiding means is a flexible sheet, the tube preferably extends along the middle part of the sheet. The term “middle part” of the sheet is defined as the middle part of the sheet seen in the transverse direction of the sheet and extending between the distal end and proximal end of the sheet.

The layers of the laminated sheet may be joined together by e.g. adhesive, pressure, welding or heat.

The laminated sheet may comprise at least three layers, including a transparent upper layer, a transparent bottom layer, and a third middle layer that is heart shaped and has an appropriate visible color. The purpose of the heart shaped third layer is to provide the sheet with an appealing vision appearance.

For the embodiments where the flexible guiding means is a flexible sheet, it is preferred that the entire inner side (or at least an appropriate part thereof) of the sheet is provided with an appropriate adhesive to releasable adhere the sheet to an insertion unit, and/or that at least a part of the outer side of the sheet is provided with an appropriate lubricant (such as a lubricant coating).

In a preferred embodiment, the artificial insemination device comprises a flexible sheet and a longitudinal tube that is secured to and extends along the sheet between the distal end and proximal end of the sheet, such as along the middle part of the sheet, where the sheet is shaped and dimensioned with a first transverse section, a second transverse section, and a third transverse section located between the first section and the second section when seen in the longitudinal length of the sheet, where the transverse width of the third section is smaller than the transverse width of each of the first section and second section. In this way, the first section and the second section of the sheet is able to wrap around an insertion unit to a higher degree than the third section. The first section is closer to the distal end of the sheet than third section of the sheet. The distal end of the first section may be the distal end of the sheet, and the proximal end of the second section may be the proximal end of the sheet. The sheet of such a device is preferably suitable for being attached to a finger when the transverse width of the third section is 15-35 mm (such as 30 mm) and each of the first and second section has a transverse width of 40-60 mm (such as 48 mm).

Such a device is particular suitable for being attached to an insertion unit in the form of a finger, where the sheet is attached to the finger with the third section of the sheet being positioned at the proximal interphalangeal joint of the finger. Each of the first section and the second section of the sheet will wrap around the finger to a higher degree than the third section. The advantage thereof is that sufficient attachment of the sheet to the finger is obtained, while bending of the finger at the proximal interphalangeal joint is only hindered to a small degree by the sheet. Such a sheet is preferably dimensioned so that when the middle part of the sheet is attached to the underside of a finger, the upper side of the proximal interphalangeal joint is be left uncovered or mainly uncovered by the sheet, so that the attached sheet does not stretch much when the finger bends.

In an alternative version of the embodiment described above, where the distal end of the first section is not the distal end of the sheet, the sheet comprise a fourth transverse section extending from the distal end of the sheet towards the first section. The fourth section has a longitudinal length (e.g. 0.5-1 cm) and a transverse width (e.g. about 3 cm or less, such as 1.5 cm or 1 cm) that is small enough to leave the underside of the fingertip uncovered by the sheet when the middle part of the sheet is secured to the upper side of a finger. The sense of touch of a finger is highest at the underside of the fingertip, which means that a device with a flexible sheet having such a fourth section in such a position on a finger, makes it easy for the user to guide the distal end of the tube towards the cervix entrance.

The thickness of a finger decreases in the direction towards the distal end of the finger (fingertip), and it is thus preferred that the transverse width of the first section is slightly smaller than the transverse width of the second section to such an extent that when the sheet is wrapped around a finger with the third section at the proximal interphalangeal joint, the first section and the second section extend substantially equally around the finger.

Such a flexible sheet may be shaped as a Christmas tree, where the fourth section is closest to the distal end of the sheet followed by the first section, the third section, and ending with the second section at the proximal end of the sheet, where the second transverse section has a larger transverse width than the first section, and the third transverse section has a smaller transverse width than each of the first section and second section. It is also within the scope of the present invention, that the first section and second section may have the same transverse width, or that the first section may have a larger transverse width than the second section. Different shapes of the sheet than the Christmas tree shape is also within the scope of the present invention.

For the embodiments of the present invention where the guiding means is a flexible sheet, the sheet may be shaped and dimensioned, such as by having a longitudinal length of about 4-6 cm along its longest longitudinal part and a transverse width of about 4-6 cm along its widest transverse part, so that its longitudinal length corresponds to the distance from the fingertip to the proximal interphalangeal joint of the middle finger of an adult human (some adults have short finger and some have longer fingers), so that the sheet may be attached to the finger where it only extends from the fingertip to the proximal interphalangeal joint, i.e. without covering the proximal interphalangeal joint or only covering a small part of the proximal interphalangeal joint.

In a further embodiment of the present invention, the flexible guiding means can be at least two wrapping strips connected to the tube and placed spaced apart along the tube, where each wrapping strip is arranged for being wrapped tightly about the insertion unit to form a closed loop and is provided with locking means for maintaining the strip in the wrapped position on the insertion unit so that the strip is releasable attached to the insertion unit.

Each of the at least two wrapping strips preferably extends substantially transverse in relation to the longitudinal length of the tube so that the each wrapping strip, when attached to the insertion unit, extends about the insertion unit in a direction that is substantially transverse in relation to the direction from the proximal end to the distal end of the insertion unit.

The locking means of the wrapping strips can be an adhesive material provided on the inner side at one end of each wrapping strip, or more preferably on the entire inner side of each wrapping strip, so that the user can wrap each strip about the insertion unit and back onto itself to lock the strip in a tightened position around the insertion unit.

The wrapping strips are preferably made of plastic, silicone, polyurethane and/or latex. Each wrapping strip may consist of two layers of film, e.g. plastic film, joined on top of each other with the tube located between the layers. The two layers may be joined by means of welding.

In a still further embodiment of the present invention, the flexible guiding means can be a narrow strip with a substantial rectangular shape, where the tube is connected to the inner side of said strip along the longitudinal length of said strip, and the inner side of said strip is provided with an adhesive material for releasable attaching said strip to an insertion unit.

The transverse width of said strip is limited to such a degree that said strip covers an area of the insertion unit that is a small as possible while providing an adhesiveness that is sufficient to substantially maintain said strip in the intended position on the insertion unit. In other words, said strip is narrow due to its limited transverse width and is not intended to be wrapped about an insertion unit.

The minimum required transverse width of said strip depends on such parameter as the strength of the adhesive material provided on the inner side of said strip, the nature of the outer surface of the chosen insertion unit, and the shape/dimensions of the tube.

The embodiment with the narrow strip is especially suitable for use with an insertion unit in the form of a finger, where a reduction in the area covered by the narrow strip increases the sense of touch of the user, which for some users is desirable.

The narrow strip is preferably made of silicone, polyurethane, latex and/or plastic.

An advantage of both the wrapping strips and the narrow strip is that both types of guiding means, when used with an insertion unit in the form of a finger of the user, highly reduces the area of the finger being covered by the guiding means and thus highly increases the sense of touch of the user.

For the embodiments according to the present invention where the guiding means is a flexible sheet, at least two wrapping strips or a narrow strip, the adhesive material provided thereon for attachment of the insemination device to an insertion unit in the form of a finger, penis or another body part of the user is preferably a hydrocolloid adhesive, an acrylic adhesive or other similar adhesives that is substantially non-reactive with the patient's tissue. A hydrocolloid adhesive absorbs moisture and lets the skin breathe so that the guiding means does not detach from an insertion unit (such as a finger) when used in a humid or wet environment (such as a vagina).

For the embodiments of the present invention where the guiding means is a flexible sheet, at least two wrapping strips or a narrow strip, it is preferred that at least the part(s) of the inner side of the sheet provided with adhesive material is/are covered by a protective cover (such as a protective film), where the cover is removed by the user before attaching the sheet to the insertion unit. The purpose of the detachable protective cover is to preserve the adhesive strength of the adhesive material during storage of the insemination device. The adhesive material is preferably adapted so that its adhesiveness after a single use is reduced to such a degree that repeated adherence to a finger is not possible, as repeated use involves the risk of contamination and possible infections.

The protective cover may be flexible, but it is preferred that it is inflexible or substantially inflexible, so that the flexible sheet is kept in a flat state during transport and until the protective cover is removed. It is easier to detach the protective cover and the sheet from each other if they initially are in a flat state. The protective cover can be a release liner, such as a silicone topped backing paper with a silicone top layer.

The insemination device of the present invention is preferably manufactured as an insemination kit in a sterile package (such as a plastic bag), where the kit comprises the longitudinal tube and the flexible guiding means and possibly a pumping mechanism, where the tube can be an integral part of the guiding means or the tube and the guiding means can be separate parts intended to be connected to each other by the user. In such a kit, the part(s) of the guiding means provided with adhesive material is/are advantageously covered by a protective cover (such as a protective film) to be removed by the user before attaching the guiding means to the insertion unit.

The present invention also relate to a method for performing artificial insemination of a subject using the insemination device or the insemination kit according to the present invention, where the method comprises:
drawing up sperm into the tube via the distal end of the tube,
attaching or securing the insemination device to an appropriate insertion unit by means of the guiding means,
inserting the insertion unit with the attached insemination device into the vagina of the subject,
ejecting at least some of the sperm from the distal end of the tube, and
removing the insertion unit with the attached insemination device from the vagina.

The method may further comprise that the distal part of the tube extending beyond the distal end of the insertion unit is cut off before inserting the insertion unit into the vagina.

Where the insemination device comprises a connector with an outlet permanently attached to the distal end of the tube and an inlet adapted to be connected to a sperm straw (such as an inlet with an opening shaped as a truncated cone tapering in the direction toward the outlet of the connector), the method of the present invention further comprises the steps of connecting the connector inlet to a sperm straw before sperm is drawn up into the tube, and afterwards removing the connector from the tube by cutting through (e.g. using scissors) the distal end of the tube next to the connector.

Where the guiding means is a flexible sheet provided with a releasable attachment means in the form of an adhesive provided on at least a part of the inner side of the sheet (or the entire inner side of the sheet), and where at least the part(s) of the sheet that is provided with the adhesive material is/are covered by a protective cover, the method of the present invention further comprises the step of removing the protective cover from the sheet before the sheet is attached to a insertion unit.

The present invention can also be used for vaginal or rectal douching, or for the administration of liquid medicine inside the vagina or the rectum.

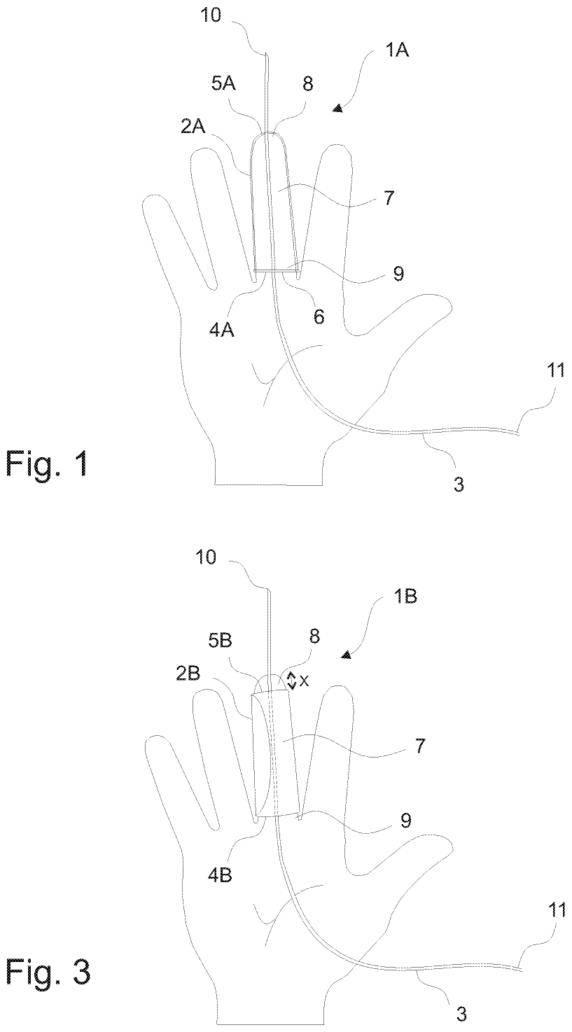

The invention will be explained in greater detail below, describing only exemplary embodiments of the artificial insemination device with reference to the FIGS. 1-18, where

FIG. 1 shows a first embodiment of the insemination device where the guiding means is a hollow tubular sleeve and the device is attached to a finger,

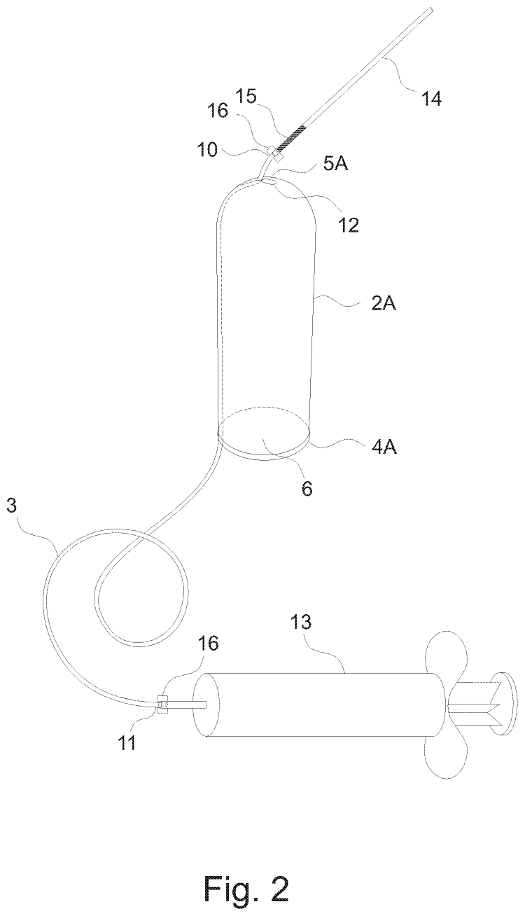

FIG. 2 shows the same device as shown in FIG. 1, where the device has been connected to a sperm straw and a syringe,

FIG. 3 shows a second embodiment of the insemination device where the guiding means is elastic flexible sheet and the device is attached to a finger,

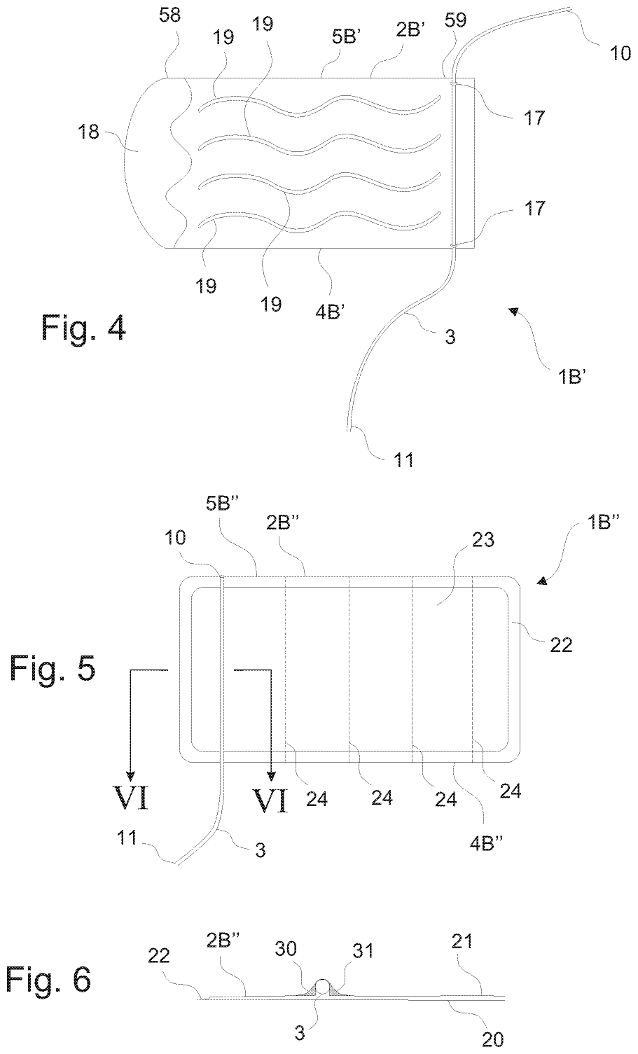

FIG. 4 shows a third embodiment of the insemination device where the guiding means is a flexible sheet,

FIG. 5 shows a fourth embodiment of the insemination device where the guiding means is a flexible sheet,

FIG. 6 shows an enlarged cross-sectional view along a section of the sheet shown in FIG. 5,

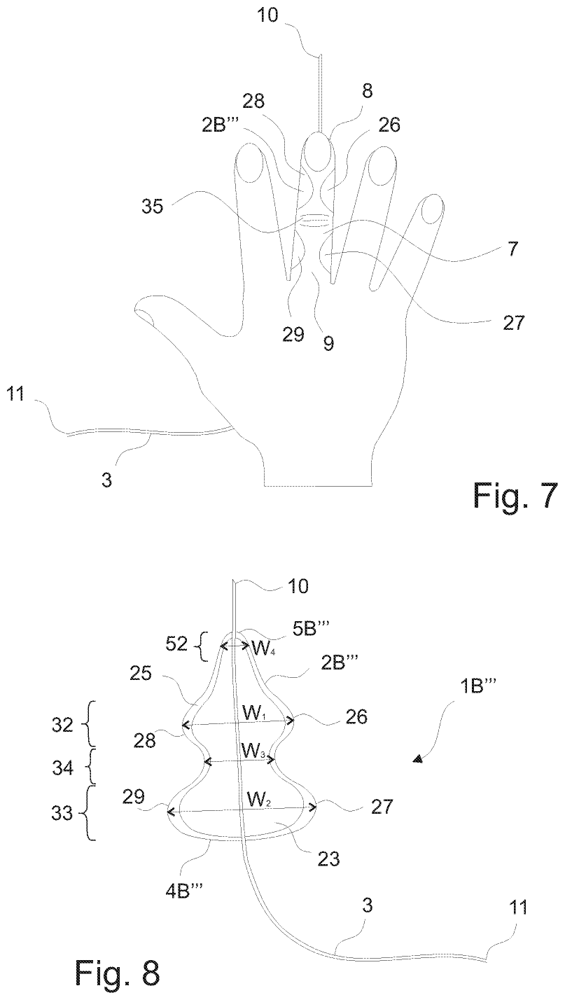

FIGS. 7 and 8 show a fifth embodiment of the insemination device where the guiding means is a flexible sheet,

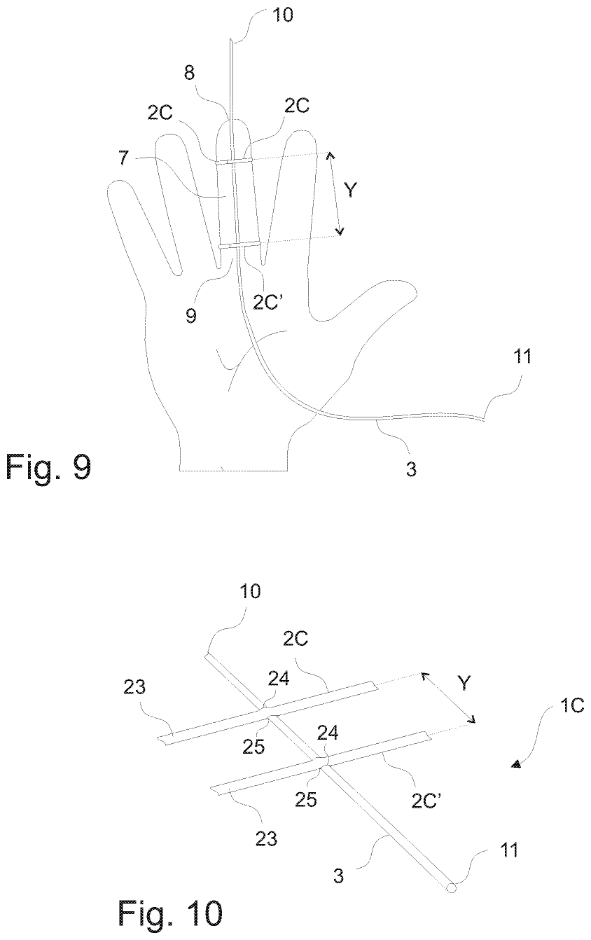

FIGS. 9 and 10 show a sixth embodiment of the insemination device where the guiding means is two wrapping strips,

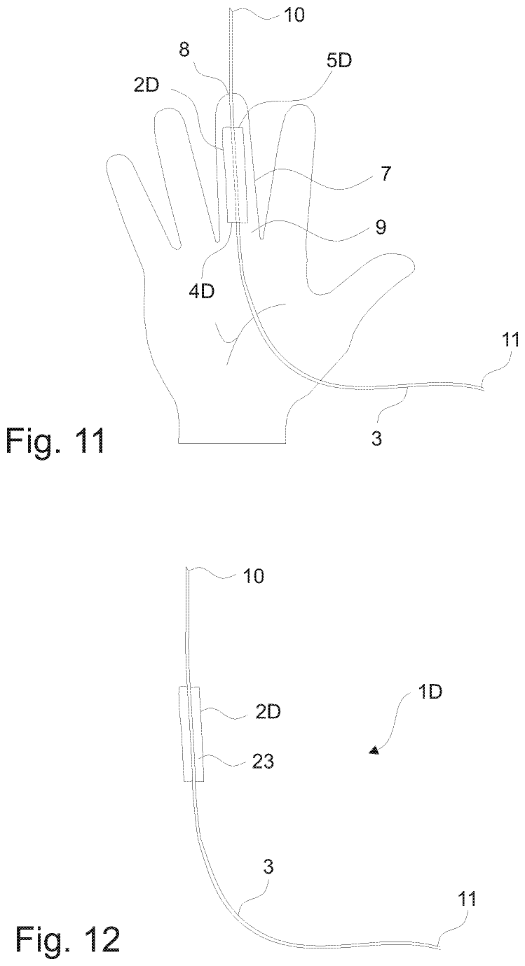

FIGS. 11 and 12 show a seventh embodiment of the insemination device where the guiding means is narrow strip,

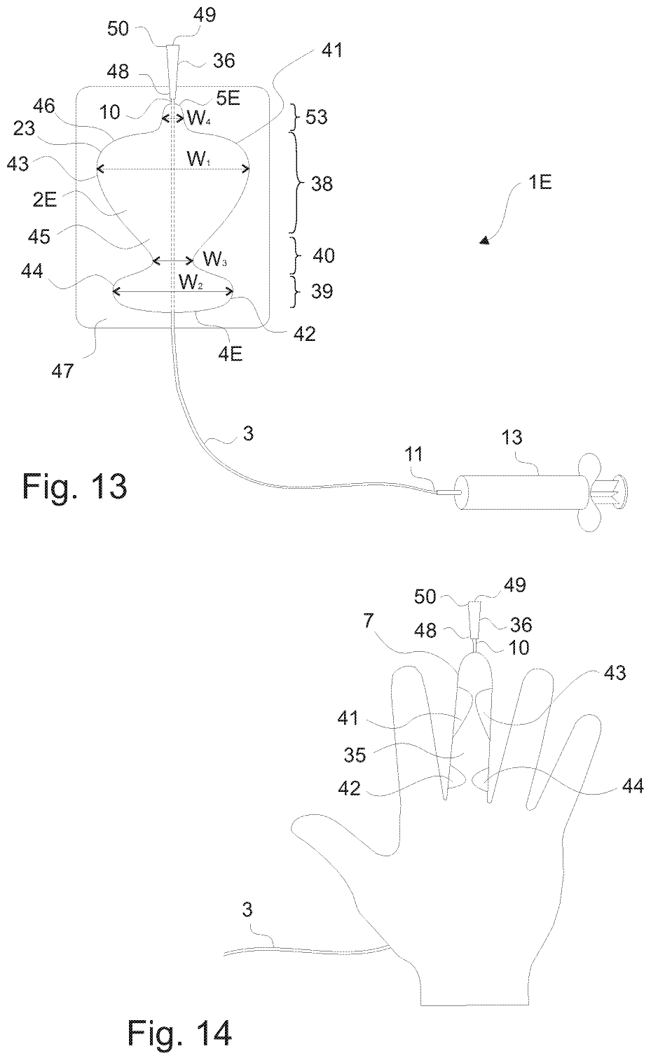

FIGS. 13-15 shows an eights embodiment of the insemination device where the guiding means is a sheet,

FIGS. 16-17 shows an ninth embodiment of the insemination device where the guiding means is a sheet, and

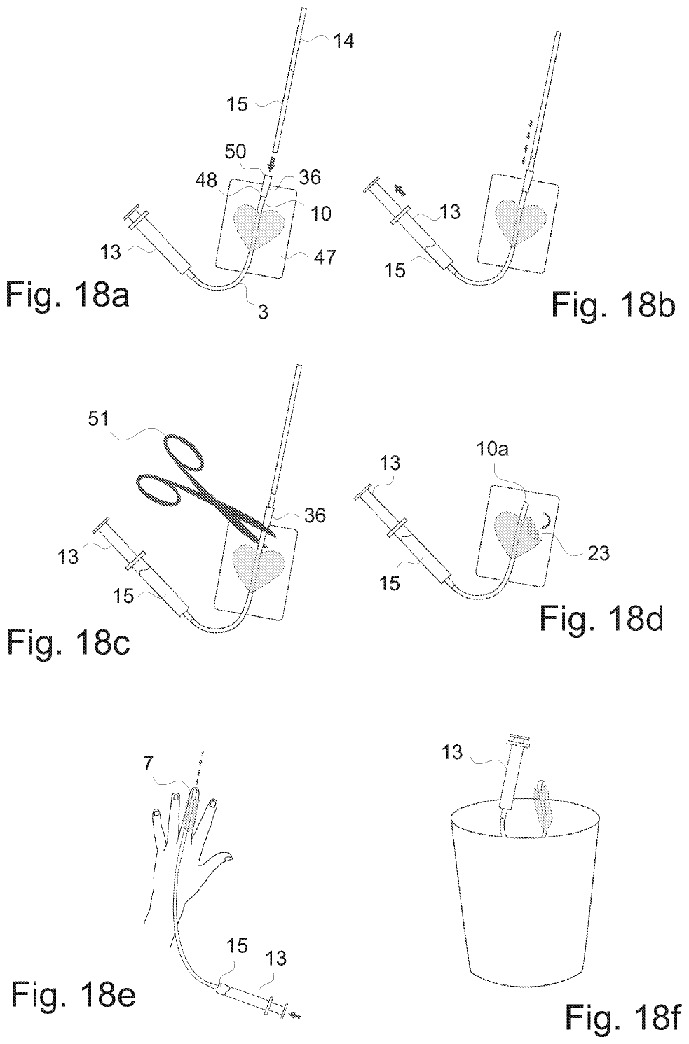

FIGS. 18a-18f illustrates the steps of using an insemination device where the guiding means is a sheet.

The insemination devices according to the figures are shown and described as being attached to a finger. However, it is within the scope of the present invention that the insemination device can be attached to other types of insertion units, in which case e.g. the shape/dimensions of the guiding means and/or the position of the tube in relation to the guiding means might very well be different than the embodiments shown in the figures.

The insemination device 1A shown in FIG. 1 consists of a hollow tubular sleeve 2A and a tube 3 connected thereto. The proximal end 4A of the sleeve 2A has an opening 6. An insertion unit in the form of a finger 7 of a user has been introduced into the sleeve 2A through said opening 6 so that the distal end 5A of the sleeve 2A is located at the distal end 8 of the finger 7, and the proximal end 4A of the sleeve 2A is located at the proximal end 9 of the finger 7. The tube 3 is embedded into the material of the sleeve 2A along the longitudinal length of the sleeve 2A, and extends beyond both the proximal end 4A and distal end 5A of the sleeve 2A. The releasable attachment means that secures the sleeve 2A to the finger 7 is primarily the friction between the sleeve 2A and the finger 7.

FIG. 2 shows the same insemination device 1A as the one shown in FIG. 1, where it is possible to see that the distal end 5A of the sleeve 2A is provided with an air hole 12 that allows air to escape from the interior of the sleeve 2A during insertion of e.g. a finger 7 as shown in FIG. 1 into the sleeve 2A.

In order to illustrative how an insemination device of the present invention can be connected to a pumping mechanism of the present invention, the proximal end 11 of the tube 3 in FIG. 2 is connected with a syringe 13.

In order to illustrate how an insemination device of the present invention can be connected to a sperm straw containing sperm, FIG. 2 shows the distal end 10 of the tube 3 being attached to a sperm straw 14 containing sperm 15. Each of the syringe 13 and the sperm straw 14 is secured to the tube 3 by means of a security tape 16 intended to be wrapped about the distal end 10 of the tube 3 and the end of the sperm straw 14.

The insemination device 1B shown in FIG. 3 consists of a flexible sheet 2B having a substantially rectangular shape and a tube 3 attached thereto. The sheet 2B is wrapped about a finger 7 of a user and the sheet 2B overlaps itself. The sheet 2B is held in place on the finger 7 by means of a releasable attachment means in the form of an adhesive coating (not shown) provided on the inner surface of the sheet 2B, i.e. the side of the sheet 2B that faces toward the finger 7. It is most practical to wrap the sheet 2B about the part of the finger 7 that has substantially the same diameter, which is the reason why the distal end 5B of the sheet 2B is at a small distance X from the distal end 8 of the finger 7. This also leaves the fingertip not covered by the sheet 2B.

The sheet 2B shown in FIG. 3 may be a laminate consisting of two layers of film joined on top of each other, where the tube 3 is located between the layers.

The insemination device 1B′ shown in FIG. 4 consists of a flexible sheet 2B′ with a substantially rectangular shape and a tube 3 connected to the inner surface of the sheet 2B′ by means of two attachment strips 17. The inner surface of the sheet 2B′ is provided with releasable attachment means in the form of a continuous band 18 of adhesive material and four strips 19 of friction generating material. The band 18 is located at one transverse side 58 of the sheet 2B′ and the tube 3 is located at the opposite transverse side 59 of the sheet 2B′. The band 18 extends from the proximal end 4B′ to the distal end 5B′ of the sheet 2B′. Each of the strips 19 extends in the transverse direction in a wave-like style, so that the strips 19 will be extending about the insertion unit when the sheet 2B′ is attached to an insertion unit during use of the device 1B′.

The insemination device 1B′ can be attached to an insertion unit, such as a finger 7, with a circumference that is smaller than the transverse width of the sheet 2B′ by wrapping the sheet 2B′ around the insertion unit starting with the transverse side 59 of the sheet 2B′ where the tube 3 is attached and ending with the transverse side 58 of the sheet 2B′ where the continuous band 18 is, so that at least a part of the sheet 1B′ with the continuous band 18 can adhere onto the sheet 2B′ itself and thereby maintaining the sheet 2B′ in the wrapped position.

The insemination device 1B″ shown in FIGS. 5 and 6 consists of a flexible sheet 2B″ and a tube 3. The sheet 2B″ consists of two plastic layers 20,21 joined on top of each other with a continuous welded portion 22 extending along the circumferential edge of the sheet 2B″. The tube 3 is located between the two layers 20,21, and in order to obtain a sheet thickness that does not change too abruptly at the location of the tube 3, the thickness of one 21 of the layers varies at the location of the tube 3 as illustrated by the hatched areas 30, 31 in FIG. 6. The two layers 20,21 of the embodiment shown in FIGS. 5 and 6 adjoin each other in the parts of the sheet without the tube, but are shown spaced apart for illustrative purposes.

The inner surface of the sheet 2B″ shown in FIGS. 5 and 6 is provided with releasable attachment means in the form an adhesive coating 23 for attachment of the device 1B″ to the finger 7. The sheet 2B″ is perforated along four lines 24, where each line 24 extends from the proximal end 4B″ to the distal end 5B″ of the sheet 2B″.

The proximal end 11 of the tube 3 extends beyond the proximal end 4B″ of the sheet 2B″, whereas the distal end 10 of the tube 3 is at the distal end 5B″ of the sheet 2B″. Thus, the embodiment shown in FIGS. 5 and 6 is intended to be wrapped about an insertion unit with the distal end 5B″ of the sheet 2B″ at the distal end of the insertion unit.

The distal end 10 of the tube 3 could instead be extending beyond the distal end 5B″ of the sheet 2B″, so that the distal tube end 10 can enter into e.g. an open container with collected sperm to be drawn up into the tube 3, where after the distal part of the tube 3 extending beyond the distal sheet end 5B″ can be cut off.

The insemination device 1B′″ shown in FIGS. 7 and 8 consists of a flexible sheet 2B′″ and a tube 3 attached to the inner surface of the sheet 2B′″ by means of an adhesive. The sheet 2B′″ consists of two layers (e.g. plastic layers) joined on top of each other with a continuous welded portion 25 extending along the circumferential edge of the sheet 2B′″. The inner surface of the sheet 2B′″ is provided with an adhesive 23 for releasable attachment of the device 1B′″ to a finger 7 as shown in FIG. 7. The sheet 2B′″ has a Christmas tree shape with a first transverse section 32 having two curved portions 26,28, a second transverse section 33 having two curved portions 27,29, a third transverse section 34 located between the first section 32 and the second section 33, and a fourth section 52, where the transverse width W₃ of the third section 34 is smaller than the transverse width W₁ of the first section 33, the transverse width W₁ of the first section 32 is smaller than the transverse width W₂ of the second section 33, and the fourth section 52 is the part of the sheet 2B′″ with the smallest transverse width W₄.

As shown in FIG. 7, the sheet 2B′″ is shaped and dimensioned so that it extends along almost the entire length of the finger 7 and does not overlap itself when wrapped about the finger 7. A significant amount of the skin on especially the top side of the finger 7 is not covered by the sheet 2B′″ in order to increase the sensitivity of the skin of the finger. FIG. 7 shows a preferred position of the sheet 2B′″ on a finger 7, where the middle part of the sheet and the tube 3 extends along the underside of the finger 7, and the third transverse section 34 is at the proximal interphalangeal joint 35. When a finger bends, it is primarily the skin at the upper side of the proximal interphalangeal joint 35 that stretches, which means that the sheet 2B′″ in the shown position on the finger 7 does not stretch much when the finger bends.

The insemination device 1B′″ shown in FIGS. 7 and 8 could be modified by locating the tube 3 between the two layers, which are joined together by e.g. an appropriate adhesive. The sheet 2B′″ could also be attached to the upper side the finger 7, so that the underside of the fingertip is left uncovered in case the user prefers to have a high degree of sense of touch.

The insemination device 1C shown in FIGS. 9 and 10 consists of two wrapping strips 2C,2C′ and a tube 3 attached to both of them. In FIG. 9 each of the wrapping strips 2C,2C′ is shown in the attached position on a finger 7 of a user where each strip 2C,2C′ overlaps itself and forms a closed loop. The strips 2C,2C′ are held in place on the finger 7 by means of an adhesive coating 23 provided on the inner surface of each strip 2C,2C′. The strips 2C,2C′ are positioned on the finger 7 with a mutual distance Y, and the tube 3 is stretched out between the wrapping strips 2C,2C′ so that the part of the tube 3 extending between the strips 2C,2C′ is as close as possible to the finger 7. The large distance Y and the stretched out tube part have the advantage that the part of the tube 3 extending between the strips 2C,2C′ are kept as close as possible to the finger 7, which decreases the probability of the tube 3 getting caught on something and thereby changes position in relation to the finger 7.

Each of the wrapping strips 2C,2C′ consists of an upper layer 24 and a lower layer 25 joined on top of each other with the tube 3 located between the layers 24,25. The two layers 24,25 may be joined by means of welding, and may be made of silicone, polyurethane or plastic.

The insemination device 1D shown in FIGS. 11 and 12 consists of a narrow strip 2D and a tube 3 connected thereto. In FIG. 11, the narrow strip 2D is attached to a finger 7 of the user and extends along a longitudinal part of the finger 7, where the part of the tube 3 that is connected to the strip 2D is located between the strip 2D and the finger 7. The narrow strip 2D is secured to the finger 7 by means of an adhesive coating 23 on the inner surface of the strip 2D, i.e. the side facing towards the finger 7 in FIG. 11.

The insemination device 1E shown in FIGS. 13-15 consists of a sheet 2E, a tube 3, a connector 36 and a pumping mechanism 13.

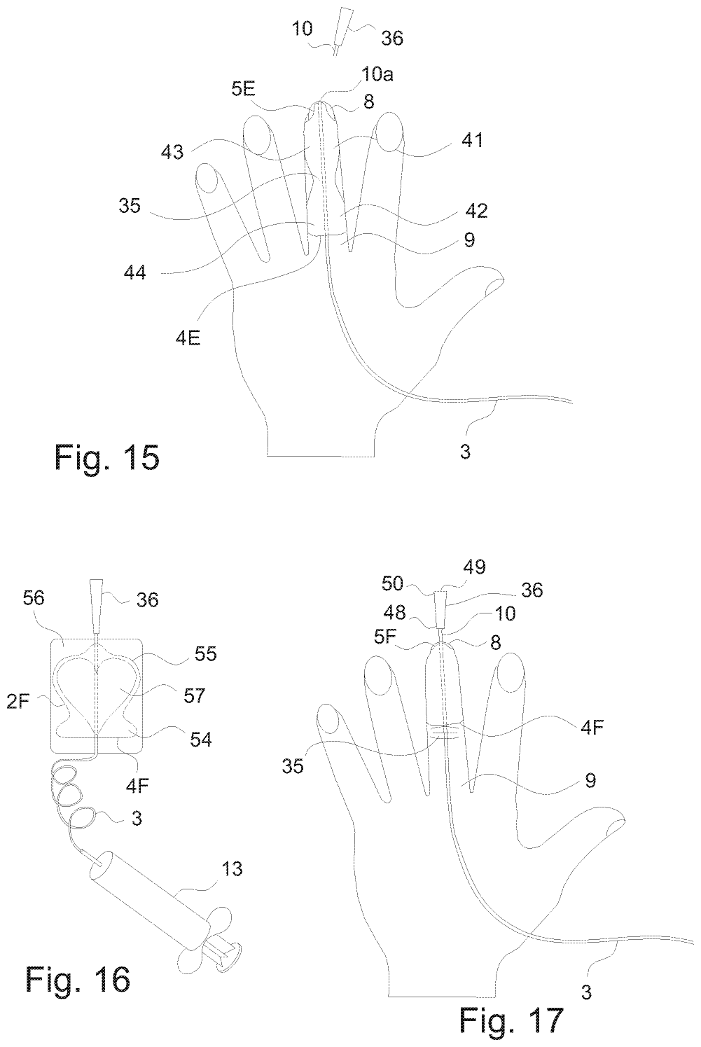

As shown in FIG. 13, the flexible sheet 2E has a first transverse section 38 with two curved portions 41,43, a second transverse section 39 with two curved portion 42,44, a third transverse section 40, and a fourth transverse section 53, where the transverse width W₃ of the third section 40 is smaller than the transverse width W₁, W₂ of both the first section 38 and second section 39, and the transverse width W₄ of the fourth section is small enough (e.g. about 1 cm) to leave the underside of the fingertip uncovered when attached to a finger 7 as shown in FIG. 15.

The sheet 2E is a laminate comprising a first layer 45 and a second layer 46 with same shape and joined on top of each other. The tube 3 is connected to the sheet 2E by being secured between the two layers 45,46. The inner side of the sheet 2E (i.e. the outer side of the second layer 46) is provided with an adhesive 23, and the adhesive strength of the adhesive 23 is preserved by the detachable protective cover 47.

The distal end 10 of the tube 3 is permanently attached to the outlet 48 of the connector 36, and the proximal end 11 of the tube is permanently attached to a pumping mechanism 13. The opening 49 of the inlet 50 of the connector 36 has the shape of a truncated cone that tapers in the direction toward the outlet 48 of the connector 36.

The device 1E is shaped and dimensioned to be especially suitable for being attached to a finger with the third transverse section 40 positioned at the proximal interphalangeal joint of the finger. FIGS. 14 and 15 shows a preferred position of the sheet 2E on a finger 7 of a hand, where the third transverse section 40 is positioned at the proximal interphalangeal joint 35 of the finger 7, the distal end 5E of the sheet 2E towards the distal end 8 of the finger 7, and the proximal end 4E of the sheet 2E towards the proximal end 9 of the finger 7. The central part of the sheet 2E is on the upper side of the finger 7 and extends to almost the very distal end 8 of the finger. The underside of the fingertip is left uncovered, which is advantageous as it is this part of a fingertip that has the highest sense of touch.

In FIG. 15, the distal end 10 of the tube 3 (and thus also the connector 46) has been cut off at the proximal end 5E of the sheet 2E, which means that the new distal end 10a of the tube 3 ends at the distal end 8 of the finger.

The insemination device 1F shown in FIGS. 16 and 17 is the same as the device 1E shown in FIGS. 13-15, except that the shape/dimensions of the first layer 54 and second layer 55 of the sheet 2F are different that the corresponding layers 45,46 of the sheet 2E, that the shape/dimensions of the protective cover 56 is different than the cover 47 of the sheet 2E, and that the laminated sheet 2F comprises a third heart shaped layer 57 located between the first layer 54 and the second layer 55. The first 54 and second layer 55 may be transparent and the third layer 57 may have an appropriate visible color. The sheet 2F could be modified by removing the third layer 57.

The device 1F is shaped and dimensioned to be especially suitable for being attached to a finger 7 with the sheet 2F extending from the distal end 8 of the finger to the proximal interphalangeal joint 35 and the third transverse section 40′ positioned at the distal interphalangeal joint of the finger. FIG. 17 shows a preferred position of the sheet 2F on the middle finger 7 of a hand, where the central part of the sheet 2F extends along the upper side of the finger 7 and the proximal end 4F of the sheet 2F is just next to the joint 35. The device 1F is advantageous, as both the underside and the upper side of the proximal interphalangeal joint 35 is not covered by the sheet 2F.

FIGS. 18a-18f illustrates the steps of using an insemination device according to the present invention, where the device comprises a connector 36 and a pumping mechanism 13 that both are permanently attached to the tube 3, and where the guiding means is a sheet. The sheet is illustrated with the shape of a heart, but the illustrated use also works with a sheet shaped as e.g. the sheets shown in FIGS. 7-8 and 13-17. First step (FIG. 18a): a sperm straw 14 is attached to the inlet 50 of the connector 36. Second step (FIG. 18b): sperm 15 is sucked from the sperm straw 14 into the tube 3 by operating the pumping mechanism 13. Third step (FIG. 18c): the connector 36 is removed from the device by cutting through the distal end 10 of the tube 3 next to the outlet 48 by means of scissors 51, where after the new distal end 10a of the tube 3 extends somewhat beyond the distal end of the sheet. Fourth step (FIG. 18d): the sheet is detached from the protective cover 47, whereby the adhesive 23 on the inner side of the sheet is exposed. Fifth step (FIG. 18e): the sheet is attached to a finger 7 by wrapping it around the finger with the inner side of the sheet facing towards the finger 7, where after the device is ready for being inserted into the vagina and ready for pumping sperm 15 out of the new distal end 10a of the tube 3 by operating the pumping mechanism 13. Sixth step (FIG. 18f): the device is thrown away after use.

For the embodiments shown in FIGS. 1-4 and 7-12, it is intended that the tube 3 should extend beyond the distal end 8 of the finger 7 so that the user conveniently can introduce the distal end 10 of the tube 3 into a portion of sperm in e.g. a cup or a vial in order to draw up sperm into the tube 3 via the distal end 10 of the tube 3. In order to drawn up the sperm, a pumping mechanism such as the syringe 13 shown in FIG. 2 can be attached to the proximal end 11 of the tube 3 and may be secured thereto by means of a security tape 16 such as shown in FIG. 2. The sperm 15 is preferably drawn up into the tube 3 after the device 1A, 1B, 1B′, 1B′″, 1C, 1D has been attached to the finger 7, so that any force applied to the tube 3 during said attachment does not unintentionally eject sperm 15 from the tube 3.

Once the sperm has been drawn up into the distal end 10 of the tube 3 in any of the embodiments shown in FIGS. 1-4 and 7-12, the part of the tube 3 extending beyond the distal end 8 of the finger 7 can be cut off, so that the new distal end of the tube 3 is at the distal end 8 of the finger 7. By doing this, the user is able to position the new distal end of the tube 3 at the cervix entrance by just positioning the distal end 8 of the finger 7 at the cervix entrance.

Once the insemination device 1B″ shown in FIGS. 5 and 6 has been wrapped about an insertion unit, the distal end 10 of the tube 3 could be connected to e.g. sperm straw with sperm and the proximal end 11 of the tube 3 could be connected to e.g. a syringe, so that the syringe can be operated in order to draw up sperm into the tube 3 before the actual insemination and subsequently eject sperm through the distal end 10 of the tube 3 during the actual insemination.

1-18. (canceled)

19. An artificial insemination device for depositing sperm at the cervix entrance of a subject, the device comprises
a longitudinal tube for accommodating sperm, and
a flexible guiding sheet connected to the tube, where the guiding sheet is arranged for being releasable attached to an insertion unit in order to facilitate at least partly insertion of the tube into the vagina by the insertion unit, wherein
the material, shape and dimensions of the sheet are of a kind that allows the sheet to be wrapped at least partly about the insertion unit, and
the sheet is releasably attached to the insertion unit.

20. The artificial insemination device according to claim 19, wherein the tube is an integral part of or embedded into the guiding sheet.

21. The artificial insemination device according to claim 19, wherein the length of the tube and the shape/dimensions of the guiding sheet are adapted so that the tube extends from a distal end of the guiding sheet to a proximal end of the guiding sheet, preferably beyond the proximal end of the guiding sheet and/or beyond the distal end of the guiding sheet.

22. The artificial insemination device according to claim 19, wherein the sheet comprises a first transverse section, a second transverse section, and a third transverse section located between the first section and the second section when seen in the longitudinal length of the sheet, where the transverse width of the third section is smaller than the transverse width of each of the first section and second section.

23. The artificial insemination device according to claim 19, wherein the sheet comprises a fourth transverse section extending from the distal end of the sheet toward the first section, where the fourth section has a longitudinal length of 0.5-1 cm and a transverse width of 1-3 cm.

24. The artificial insemination device according to claim 19, wherein the sheet is releasably attached to the insertion unit by an adhesive material provided on at least a part of the inner surface of the sheet.

25. The artificial insemination device according to claim 24, wherein at least the part(s) of the sheet provided with said adhesive material is/are covered by a removable protective cover that is inflexible or substantially inflexible.

26. The artificial insemination device according to claim 19, wherein the tube is connected to the sheet at one of its transverse sides, and one or more bands of adhesive material is provided at the opposite transverse side of the sheet.

27. The artificial insemination device according to claim 19, wherein the sheet is releasably attached to the insertion unit by one or more strips of friction generating material provided on the inner surface of the sheet and extending substantially transverse in relation to the direction from the proximal end of the sheet to the distal end of the sheet, where the friction generating material is of a kind providing friction between the sheet and the insertion unit.

28. The artificial insemination device according to claim 19, wherein the sheet is provided with at least one perforated line extending from the proximal end of the sheet to the distal end of the sheet.

29. The artificial insemination device according to claim 19, wherein the sheet is a laminate comprising at least two layers joined on top of each other, and the tube is embedded between the two layers.

30. The artificial insemination device according to claim 19, wherein the thickness of the sheet in the proximity of the tube is adapted to obtain a smooth transition from the parts of the sheet without the tube to the part of the sheet where the tube is located.

31. The artificial insemination device according to claim 19, wherein the device comprises a connector with an inlet and an outlet, where the inlet is adapted to ensure a fluid tight connection with a sperm straw, and the outlet is adapted to ensure a fluid tight connection with the distal end of the tube.

32. The artificial insemination device according to claim 31, wherein the outlet of the connector is permanently attached to the distal end of the tube.

33. The artificial insemination device according to claim 31, wherein said inlet has the shape of a truncated cone that tapers in the direction toward said outlet.

34. The artificial insemination device according to claim 19, wherein said device comprises a pumping mechanism permanently connected to the proximal end of the tube in a fluid tight manner.

35. Use of the artificial insemination device according to claim 19 for administration of liquid medicine inside the vagina or the rectum.

36. A Kit comprising:
the device according to claim 19, and
a sterile package with a compartment for accommodating the device.
